(19) United States
(12) Patent Application Publication
(10) Pub. No.: US 2020/0051678 A1
(43) Pub. Date: Feb. 13, 2020

(54) Calamity Averse Prescription Method and Apparatuses

(71) Applicant: Allen Hurt, Hobbs, NM (US)

(72) Inventor: Allen Hurt, Hobbs, NM (US)

(21) Appl. No.: 16/101,825

(22) Filed: Aug. 13, 2018

(51) Int. Cl. G16H 20/10; G16H 50/30

(52) U.S. Cl. CPC G16H 20/10; G16H 50/20; G16H 50/30

(57) ABSTRACT

A method and apparatuses for executing a method for issuing a prescribing professional pre-approved pharmaceutical prescription for a patient for treating a relatively minor condition resulting from a common, acute simple pathological process. The method providing a patient or user with a series of advisories in order for the patient to determine that they are not experiencing symptoms indicative of a possible emergent, urgent or complicated condition, a choice of alternate pharmaceutical therapeutics and immediate prescription transmission to a pharmacy of their choice.

### FIELD OF THE INVENTION

This application pertains to automated apparatuses and method for limited prescribing of selected pharmaceutical therapeutics for a limited number of the most common, acute, simple ailments seen by healthcare professionals.

### BACKGROUND OF THE INVENTION

Traditionally, healthcare patients have limited options in obtaining pharmaceutical prescriptions when experiencing common, acute, simple ailments. This is despite the fact that simple, safe pharmaceutical therapies make up for 5% of all annual prescriptions. Under current practice, the only way to obtain a safe, effective prescription for a patient's simple condition is to visit a provider in a physician practice, an urgent care center, an emergency room or on-line, just to obtain a prescription for a commonly prescribed medication. For patients experiencing simple ailments it is a frustrating, expensive, time consuming experience having to endure travel to a healthcare facility, long wait times due to their lower priority condition, being exposured to other patients' sicknesses, and then to incur the relatively high fees and co-payments for an, at times, unnecessarily complicated work up, then having to wait at the pharmacy for the prescription to be filled.

Health care providers spend a disproportionate amount of time diagnosing common, acute, simple ailments that take away from delivering quality healthcare to more serious patients, where their time is better used.

The traditional differential diagnostic method used by healthcare practitioners is a systematic diagnostic method used to identify the presence of a disease entity where multiple alternatives are possible. This method is essentially a process of elimination or at least a process of obtaining information that shrinks the “probabilities” of candidate conditions to negligible levels, by using evidence such as symptoms, patient history, and medical knowledge to adjust epistemic confidences in the mind of the physician.

Differential diagnosis can be regarded as implementing aspects of the hypothetico-deductive method, in the sense that the potential presence of candidate diseases or conditions can be viewed as hypotheses that physicians further determine as being true or false. Most all of the information obtained by the physician, at least initially, is via a question and answer format.

A standard of care differential diagnosis has four steps. Patient safety requires that the physician:

Gathers all information about the patient history and symptoms, and creates a list of possible diagnosis. The list can be in writing or in the physician's head, as long as he/she makes a list.

Lists all possible causes, or candidate conditions, for the symptoms. Again, this can be in writing or in the physician's head but it must be done.

Prioritizes the list by placing the most urgently dangerous possible causes at the top of the list.

“Rules out” or treats possible causes, beginning with the most urgently dangerous condition and working down the list. Rule out—practically—means use tests and other scientific methods to determine that a candidate condition has a clinically negligible probability of being the cause.

If no diagnosis remains, it means either that the physician made an error, or that the cause of the condition cannot be diagnosed.

The differential diagnosis process used is lengthy, often requires more information than the physician has access to at the time of the initial engagement with the patient, and is ideally suited towards non-simple, possible emergent, urgent and complicated conditions, which can be diagnosed using further tests and observations.

Each year, there are approximately 350 million acute visits to healthcare facilities in the U.S. About 200 million of these acute visits are for Ear, Nose & Throat (ENT) and pulmonary conditions and approximately 160 million of these are for patients aged 13 years and older. Approximately half of these are Primary Care Physician (PCP) visits and the other half encompass mostly emergency room and urgent care center visits. Yet, all of these facilities are set up to mix the simple cases in with the most serious emergent, urgent and complicated cases. Thus, the traditional approach to evaluating, assessing, diagnosing and prescribing is costly, inefficient and unnecessary. The healthcare system is getting overwhelmed in the U.S. and the United States ranks as the most expensive nations for healthcare in the world on a per capita basis.

The U.S. economy ends up footing the bill for the resultant cost of treating common, acute, simple ailments in this manner. Treatment of a simple ear ache has been known to cost $1,500.

The advent of on-line medical services promised to eliminate some of these frustrations. However, patients and insurances must still pay for a physician and the patient must still participate in the differential diagnosis process that the traditional physician model relies upon. Furthermore, on-line physician “visits” are limited in their ability to completely evaluate and assess a patient due to the limitations through video conferencing software, limitations on time, the existence of human error, inefficiency of the differential diagnosis approach and the economic pressures presented by the fee for service practice of medicine. Thus, while eliminating the travel and potential exposure to sick patients, the physician bottleneck remains.

On-line symptom checkers are automated versions of differential diagnostic models and have become available and helpful in empowering patients to begin to participate through the differential diagnosis model. However, as is the case when seen by a physician, the process can only advance through to a diagnosis after appropriate tests and observations have been made. Thus, these tools have become helpful to some patients in ruling out some of the more serious diagnoses on their own, and/or enabling the patient to ultimately have a more active participation in a dialog with a physician or healthcare provider. They do not, however, enable timely issuance of safe, effective prescriptions for common, acute, simple ailments.

As a result, these new tools do not address the almost 200 million patients per year that are experiencing only a common, acute, simple ailment and may require only a safe, effective prescription. Most of these patients have a very high degree of familiarity with their common condition, most likely having experienced it at least once or even multiple times in the past, for which they visited a healthcare facility to obtain a common prescription.

The shared qualities of common, acute and simple processes allow them to be aggregated into a niche. The nature of this straight forward niche of processes renders them open to a treatment approach that does not require the active participation of a human brain.

Therefore, what is needed in the art is a tool that enables patients experiencing a common, acute, simple ailment to be able to obtain a prescribing professional's prescription for a safe, effective prescription transmitted directly to the patient's pharmacy of choice without enduring the travel to a healthcare facility, long wait times to see a physician due to their lower priority condition, exposure to other patients' sicknesses and the relatively high fees and co-payments for an unwarranted and unnecessary differential diagnostic work up.

It is an objective of the current invention to guide patients by way of common, lay language advisories, with information for differentiating simple versus urgent conditions.

It is also an objective of the current invention to assist patients in choosing alternative medications most suitable for them.

It is also an objective of the current invention to make the guidance and assistance available at any time of day.

It is also an objective of the current invention to provide near immediate transmission of a safe and effective prescription for medication suitable for the treatment of their complaint to a patient's pharmacy of choice.

### BRIEF DESCRIPTION OF THE INVENTION

The current invention recognizes that the explanation for a large majority of the complaints brought to the primary care setting can be found in one of a very small number of common, acute, simple disease processes, and the resulting safe and effective pharmaceutical therapies that can be made available as a result of this recognition. The current invention eschews the traditional physician-centric costly, lengthy, unnecessary differential diagnosis approach to diagnosis and treatment, and substitutes in its place the innovation of empowering the patient to focus not on what “is” the explanation of the complaint, but instead on what “is not” the explanation of the complaint. This is done in the form of a series of advisories and warnings presented to the patient. These advisories and warnings address all of the possible emergent, urgent or complicated explanations for the patient's condition. If the patient concludes that any of the advisories or warnings are possibly applicable to their condition, the patient is advised to seek medical attention in a traditional primary care, urgent care or emergency medicine setting.

If, after considering the advisories and warnings, the patient feels that they are not applicable, the patient is offered prescriptive treatment options best suited to the condition. This process is completed without the participation of an active physician and can be done in a matter of minutes, any time of the day or night. It does not require a lengthy rule-in/rule-out work-up and is available to the patient wherever the patient may be, whether sick at home, at school, at work, or otherwise out and about. Because the process is standardized, there is no possibility for human error, no rush on the part of the physician to get to the next patient, no lapse of memory, no distractions.

The current invention is a new, innovative approach to the treatment of the niche of common, acute, simple diseases now seen in the clinical setting. It provides a significant improvement in quality at a significant reduction in time and cost.

The current invention comprises the elements and methods necessary to realize the described advantages in the form of apparatuses and method implemented on a server, in communication with patients, configured to issue advisories and warnings, recommend pharmaceutical therapies, generate and transmit a prescription to a pharmacy and create an electronic medical record.

Altogether, aspects of the disclosure offer heretofore unavailable cost, safety and efficiency benefits to patients. The presentation of relevant advisories and warnings to a patient or a user and near instant acquisition of a safe, effective prescription remedy when appropriate, from the safety of their location, is useful to a patient wanting to make better healthcare decisions and obtain better healthcare, rather than incurring a lengthy, potentially irrelevant differential diagnosis process, including travel, wait disease exposure and economic considerations.

### BRIEF DESCRIPTION OF THE DRAWINGS

FIG. 1 illustrates how the invention communicates with patients, pharmacies and merchant services.

FIG. 2 illustrates the physical elements of the invention.

FIG. 3 illustrates the logical elements of the invention.

FIG. 4 illustrates the flow chart of the invention's application engine.

FIG. 5 illustrates the flow chart of the invention's advisory confirmation module.

FIG. 6 illustrates the flow chart of the invention's prescription confirmation module.

FIG. 7 is an example presentation of the patient condition choices.

FIG. 8 is an example presentation of a patient-selected condition details and warning.

FIG. 9 is an example presentation of a patient-selected condition advisory.

FIG. 10 is an example presentation of a proposed pharmaceutical therapeutic.

FIG. 11 is an example illustration of a patient information input presentation.

FIG. 12 is an example illustration of a payment information input presentation.

FIG. 13 is an example prescription document.

### DETAILED DESCRIPTION OF THE INVENTION

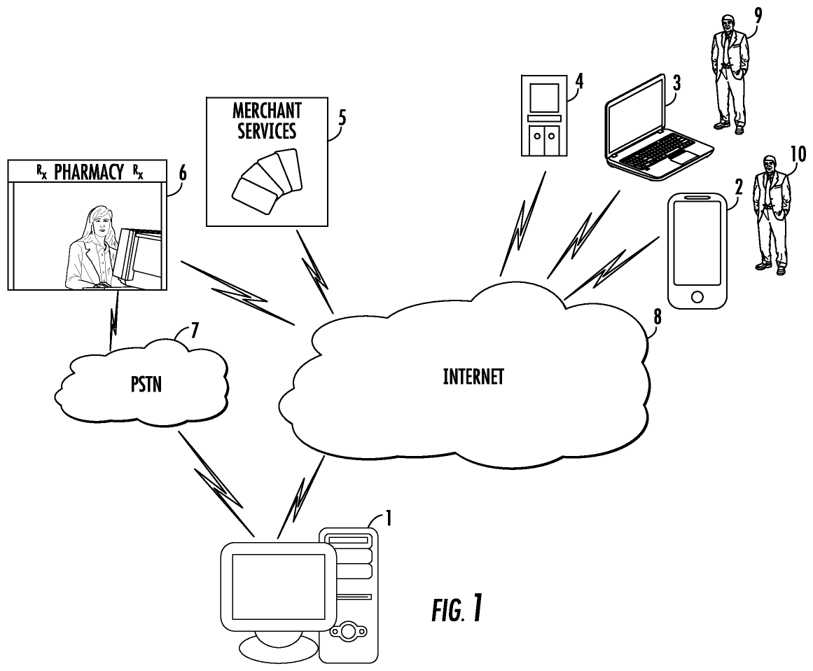

An embodiment of the current invention is shown and generally designated by the reference number 1. FIG. 1 illustrates the calamity averse prescription apparatus 1 as connected to the internet 8 for communication with patients 10, pharmacies 6 and merchant services 5. The calamity averse prescription apparatus 1 also interfaces to the public telephone switched network, or PSTN 7, for communicating via facsimile to pharmacies 6. In a preferred embodiment, the calamity averse prescription apparatus 1 is maintained at a secure location, such as a US Health Insurance Portability and Accountability Act (HIPAA) compliant data center with access and connectivity to the internet 8 and PSTN 7.

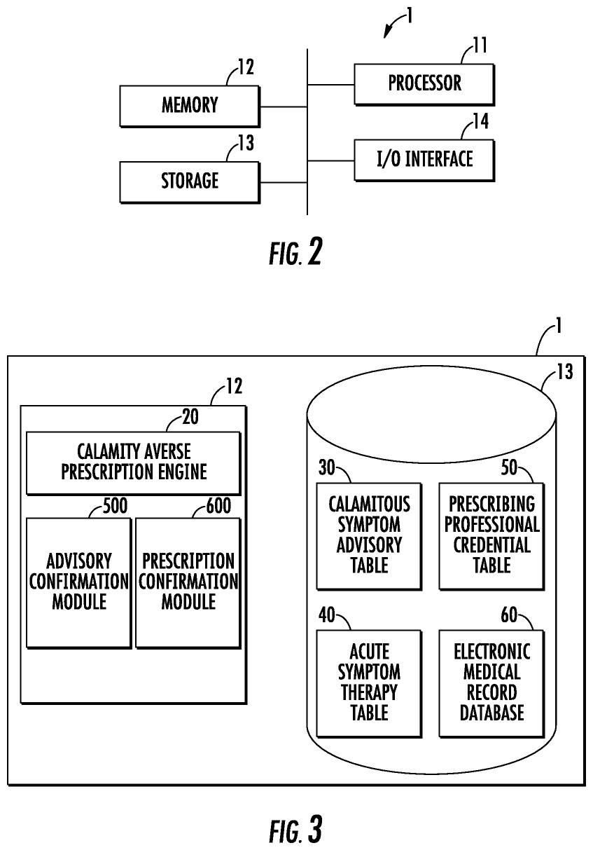

Referring the invention apparatus 1 in FIG. 2, exemplary processor 11 can be implemented as a single or plurality of a conventional microprocessor, application specific integrated circuit (ASIC), digital signal processor (DSP), programmable gate array (PGA), or the like. The calamity averse prescription apparatus 1 may also include a plurality of connected individual computer systems. The processor 11 executes the instructions that are stored in order to process data. The set of instructions may include various instructions that perform a particular task or tasks, such as those shown in the appended flowcharts. Such a set of instructions for performing a particular task may be characterized as a program, software program, software, engine, module, component, mechanism, or tool.

The memory 12 may include random access memory (RAM). The memory 12 includes application programs, OS, application data etc. The non-volatile memory 13 may include most types of computer storage devices, including, but not limited to, hard disk drives, solid state drives, floppy disks, magnetic tape and optical discs. The non-volatile memory 13 is configured to include databases, tables and apparatus configuration data. The invention apparatus 1 also includes an input/output (I/O) interface 14 for communication via the internet 8, PSTN 7 and non-transitory computer-readable media.

The calamity averse prescription apparatus 1 presents information to and receives input from user computing devices such as tablets, iPads, Mac OS computers, Windows computers, e-readers, and mobile user devices such as the iPhone, Android, and Windows Phone. The user computing devices can connect to the calamity averse prescription apparatus 1 via the internet. Patients 10 or users 9 can communicate with the calamity averse prescription apparatus 1 via any of user devices mentioned including, but not limited to smart phones 2, computers 3 of all formats, kiosks 4 and other user devices capable of hosting a web-browser for sending and receiving information to the calamity averse prescription apparatus 1. An exemplary user computing device contains a web browser or similar program, allowing in some embodiments for a secure SSL connection, and able to display HTML and CSS.

It is intentioned that a patient 10 interacts with the calamity averse prescription apparatus 1 directly using information presented by the calamity averse prescription apparatus 1 and inputting information into the calamity averse prescription apparatus 1. However, in some cases, the patient 10 is either a child under the care of an adult, or another adult is caring for an adult patient 10. In such case, a user 9 interacts directly with the calamity averse prescription apparatus 1, representing the condition of the patient 10 when interacting with the calamity averse prescription apparatus 1. Information exchange with any merchant services 5 is accomplished via standard payment interfaces and protocols commonly used in on-line purchasing carts.

FIG. 3 illustrates the logical layout of the memory and storage elements of the calamity averse prescription apparatus 1, including at least one storage device and memory, the at least one storage device is configured to comprise a calamitous symptom advisory table 30, an acute symptom therapy table 40, a prescribing professional credential table 50 and an electronic medical record database 60, the memory configured to comprise a calamity averse prescription engine 20 comprising an advisory confirmation module 500 and a prescription confirmation module 600. The software tables, database, engine and modules stored in memory and non-volatile memory of the calamity averse prescription apparatus 1 may be stored using any data structures known in the art including files, arrays, linked lists, relational database tables and the like. The apparatus 1 is capable of reading executable instructions from a non-transitory, computer readable medium.

The calamitous symptom advisory table 30 contains a limited number of patient conditions that are due to common, acute, simple pathological processes for which a patient 10 or a user 9 wishes to obtain a prescription. Examples of such include, but are not limited to, common cold, flu, cough, bronchitis, whooping cough, migraine headache, pink eye, sinusitis, tooth ache, urinary tract infection, shingles flare-up, ear ache, sore throat, urinary tract infection and sea sickness. The calamitous symptom advisory table 30 is configured to include information associated with each particular condition including expected symptoms of each condition, as well as disqualifying co-conditions that represent possible emergent, urgent or complicated conditions that warrant immediate medical attention. Included in the calamitous symptom advisory table 30 along with the disqualifying co-conditions are the risk and possible cause associated with the disqualifying co-conditions. Collectively, the disqualifying co-condition, risk and possible cause are referred to as an advisory. The advisory is associated with at least one possible emergent, urgent or complicated condition.

The acute symptom therapy table 40 contains a limited number of safe, effective pharmaceutical therapeutics most prescribed to patients experiencing common, acute, simple pathological processes. Examples of such include, but are not limited to, amoxicillin, sulfamethoxazole/trimethoprim, acyclovir, azithromycin, fluconazole, scopolamine, cephalexin, sulfacetamide ophthalmic solution, cefuroxime, oseltamivir, metronidazole, and others.

Each of the pharmaceutical therapeutics may be associated with safe and effective treatment of at least one of the common, acute, simple pathological processes in the calamitous symptom advisory table 30, and more importantly, at least one of the pharmaceutical therapeutics is associated with each one of the common, acute, simple pathological processes in the calamitous symptom advisory table 30. Included in the acute symptom therapy table 40 are the dose, dosage and re-fill information associated with each pharmaceutical therapeutic further associated with each common, acute, simple pathological process.

A prescribing professional credential table 50 contains prescribing professional information including, but not limited to, name, address phone number and national provider identifier (NPI) number, which is the required identifier for Medicare services, and is used by other payers, including commercial healthcare insurers. Collectively, this information is required on the part of a prescribing professional on issued prescriptions. Furthermore, each prescribing professional in the table is associated with at least one of the conditions in the calamitous symptom advisory table 30 and with at least one of the pharmaceutical therapeutics in the acute symptom therapy table 40.

The calamity averse prescription apparatus 1 gives the prescribing professional authority to configure which conditions and associated pharmaceutical therapeutics are associated with their name. The inclusion of the information in the prescribing professional credential table 50 allows for individual prescribing professionals to set customized and limited prescriptive conditions under which the calamity averse prescription apparatus 1 will issue safe, effective pharmaceutical therapeutics under the authority of the prescribing professional to patients experiencing common, acute, simple pathological processes.

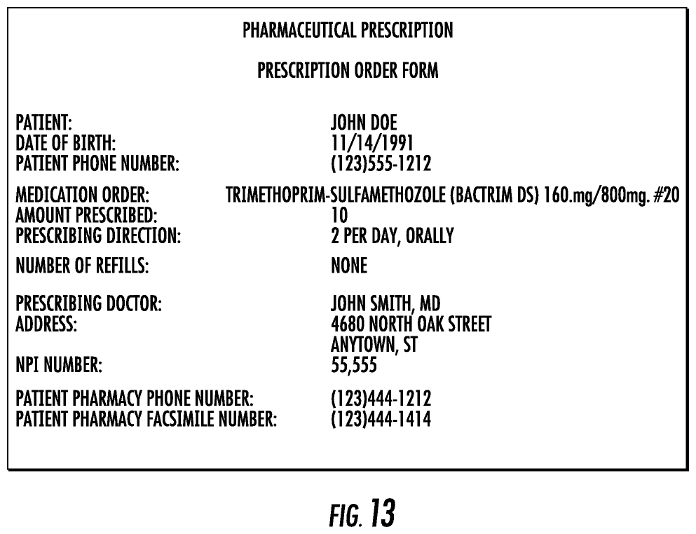

The electronic medical record database 60 contains a complete patient record of the patient's interaction with the calamity averse prescription apparatus 1, including any pharmaceutical prescription, see example FIG. 13, transmitted to a pharmacy 6. Information in the patient record in the electronic medical record database 60 also includes patient acknowledgements, condition selected, pharmacy transmittal data, patient name, phone number and date of birth, user-selected pharmacy phone and facsimile numbers, the prescribing professional's name, address and NPI number, and date and time the pharmaceutical prescription was transmitted.

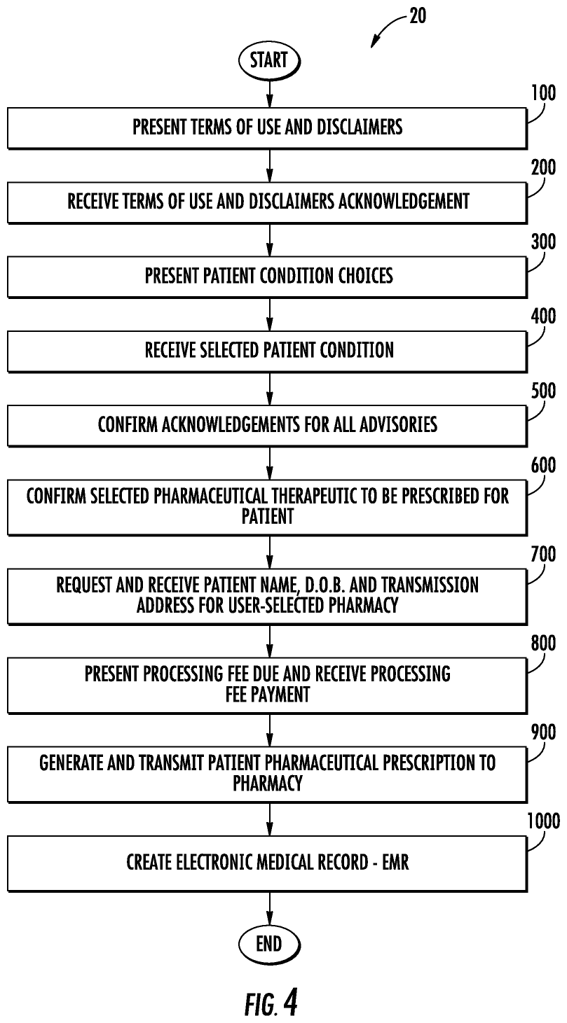

The patient 10 interacts with the calamity averse prescription apparatus 1 in the method performed by the calamity averse prescription engine 20 illustrated in FIG. 4. The method 20 receives input from the patient 10 or user 9 that the patient 10 wishes to proceed in obtaining a prescription for treatment of a common, acute, simple disease process.

In the first block 100, the patient 10 may be presented with a set of disclaimers and terms of use of the calamity averse prescription apparatus 1 by the calamity averse prescription engine 20. The terms of use serve to qualify the patient 10 and user 9 by ensuring that the user 9 is over the age of 18, that the patient is over the age of 13 and if the patient 10 is a minor, ensuring that the user 9 has the authority to make medical decisions for the minor patient 10. The disclaimers may include statements describing limitations of the calamity averse prescription apparatus 1, use of the information presented by the apparatus, lack of endorsements and other generalized instructions on use of the apparatus 1. Upon being presented with the disclaimers and terms of use of the calamity averse prescription apparatus 1 by the calamity averse prescription engine 20, the patient 10 or user 9 must then acknowledge, where the acknowledgements are received in box 200.

If the patient 10 or user 9 fails to acknowledge terms of use or disclaimers, the calamity averse prescription engine 20 will fail to advance to the next steps. In another mode, the calamity averse prescription engine 20 detects the acknowledgement failure and presents a message confirming the lack of acknowledgement to the patient 10 or user 9 and begins over.

The calamity averse prescription engine 20 then advances to box 300 where the conditions from the calamitous symptom advisory table 30 are presented to the patient 10 or user 9. An example of the presentation can be seen in FIG. 7. The patient 10 or user 9 then decides which, if any, of the limited number of patient conditions listed are those being experienced by the patient. The Patient 10 or user 9 then selects the condition for which the patient 10 desires a prescription and the calamity averse prescription engine 20 receives the choice in box 400.

If none of the listed conditions listed describe that being experienced by the patient, then the calamity averse prescription engine 20 will fail to advance to the next steps. In another mode, calamity averse prescription engine 20 detects the selection failure and presents a message confirming the lack of selection to the patient 10 or user 9 and returns control back to the calamity averse prescription engine 20 at END.

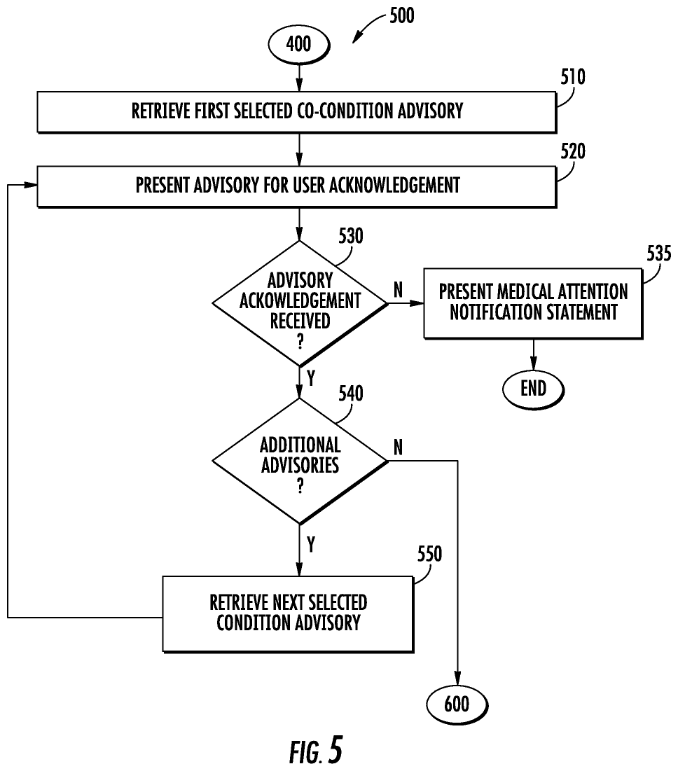

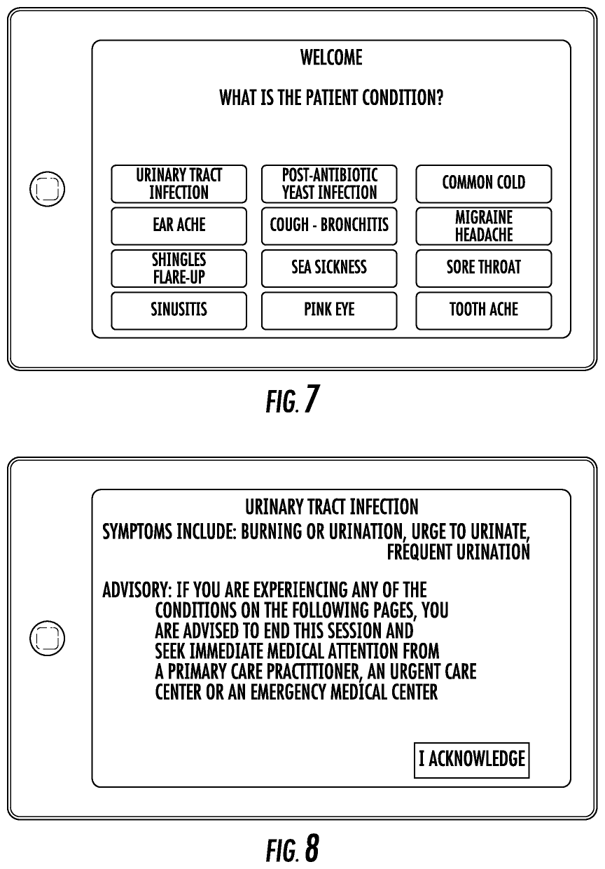

The calamity averse prescription engine 20 then advances to box 500 for confirmation of all patient condition advisories, where the calamity averse prescription engine 20 passes control to the advisory confirmation module 500, FIG. 5. In box 510 of the advisory confirmation module 500, the advisory confirmation module may optionally present to the patient 10 or user 9 a pre-advisory warning message, instructing the patient 10 or user 9 on expected symptoms associated with a condition related to a common, acute, simple pathological process. FIG. 8 exemplifies such a pre-advisory warning message presentation for a selected condition. To obtain confirmation of all patient condition advisories, the advisory confirmation module 500 at box 510 retrieves the first disqualifying co-condition that is associated with the selected condition received from the patient 10 or user 9, along with the associated risk and possible cause, collectively called the advisory, from the calamitous symptom advisory table 30.

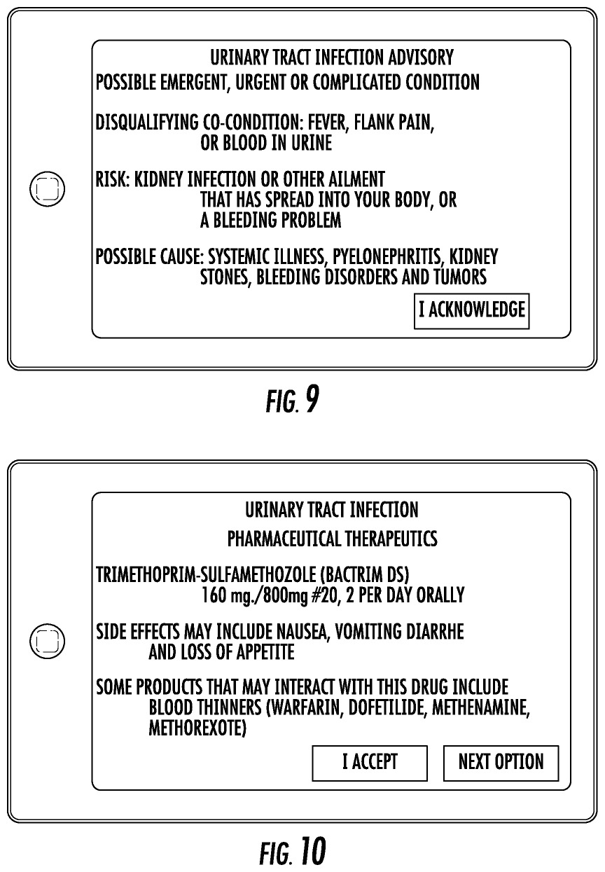

The module then advances to box 520 where the advisory from the calamitous symptom advisory table 30 is presented to the patient 10 or user 9. An example of a typical advisory presentation can be seen in FIG. 9. The patient 10 or user 9 may then assess whether or not the disqualifying co-condition, risk and possible cause in the advisory are applicable to the patient 10 condition. Empowered with this information, the patient 10 or user 9 may then acknowledge the advisory and choose to continue towards choosing a pharmaceutical therapeutic.

Once the module receives the patient 10 or user 9 advisory acknowledgement in box 530, the module determines if there are any additional advisories in the calamitous symptom advisory table 30. If so, the module retrieves the next disqualifying co-condition that is associated with the selected condition received from the patient 10 or user 9, along with the associated risk and possible cause from the calamitous symptom advisory table 30, box 550, and returns back to box 520 to present the advisory to the patient 10 or user 9 as described above. After presenting all advisories associated with the condition selected to the patient 10 or user 9 and receiving acknowledgements of those advisories from the patient 10 or user 9, the module then returns control back to the calamity averse prescription engine 20 at box 600.

If the patient 10 or user 9 fails to acknowledge any presented advisory, the calamity averse prescription engine 20 will fail to advance to the next steps. In another mode, box 535, the module detects the acknowledgement failure and presents a medical attention notification statement to the patient 10 or user 9, advising the patient 10 to seek immediate medical care, and returns control back to the calamity averse prescription engine 20 at END.

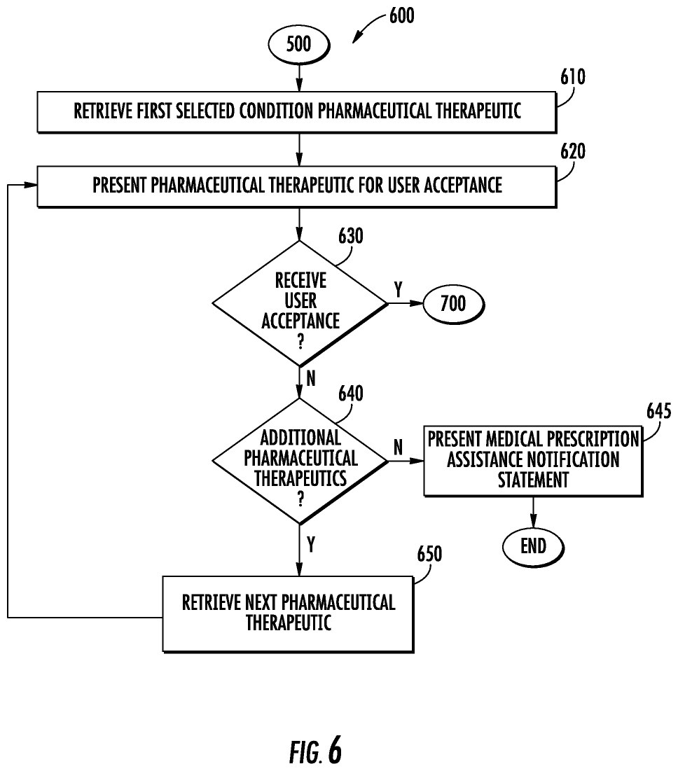

At box 600, the calamity averse prescription engine 20 passes control to the prescription confirmation module 600, FIG. 6. In box 610 the prescription confirmation module retrieves the first pharmaceutical therapeutic from the acute symptom therapy table 40 that is associated with the selected condition received from the patient 10 or user 9.

The module then advances to box 620 where the pharmaceutical therapeutic retrieved from the acute symptom therapy table 40 is presented to the patient 10 or user 9 for acceptance. FIG. 10 exemplifies such an example of the presented pharmaceutical therapeutic. The patient 10 or user 9 may then assess whether or not the presented pharmaceutical therapeutic is acceptable, and not counter-productive to the patient 10 condition. For example, the patient 10 may have a known allergic reaction to the pharmaceutical presented, in which case, the presented pharmaceutical therapeutic would not be acceptable. Empowered with this information, the patient 10 or user 9 may then accept the pharmaceutical therapeutic presented and continue towards getting a prescription issued. Once the prescription confirmation module 600 receives the patient 10 or user 9 acceptance in box 630, the module returns control back to the calamity averse prescription engine 20 at box 700.

If at box 630 the patient 10 or user 9 does not accept the presented pharmaceutical therapeutic, but instead selects the option to be offered an alternative pharmaceutical therapeutic, the prescription confirmation module 600 advances to box 640 to determine whether or not there are additional pharmaceutical therapeutics in the acute symptom therapy table 40 that are associated with the selected condition received from the patient 10 or user 9. If so, the prescription confirmation module 600 retrieves the next pharmaceutical therapeutic that is associated with the selected condition received from the patient 10 or user 9, box 650, and returns back to box 620 to present the pharmaceutical therapeutic to the patient 10 or user 9 as described above.

If, at box 640, the module 600 determines that there are no more available pharmaceutical therapeutics associated with the condition selected by the patient 10 or user 9, the module may present a medical prescription assistance notification statement advising the patient 10 or user 9 to seek immediate medical care, and returns control back to the calamity averse prescription engine 20 at END.

If the patient 10 or user 9 fails to accept either the presented pharmaceutical therapeutic or any of the alternative options, the prescription confirmation module 600 will fail to advance to the next steps. In another mode, if the patient 10 or user 9 fails to accept either the presented pharmaceutical therapeutic or any of the alternative options, the module detects the selection failure and may present a medical prescription assistance notification statement advising the patient 10 or user 9 to seek immediate medical care, and returns control back to the calamity averse prescription engine 20 at END.

After having received the patient 10 or user 9 accepted pharmaceutical therapeutic for the selected condition, the calamity averse prescription engine 20 at box 700 presents the patient 10 or user 9 with a request for the patient name, date of birth and phone number, and the phone number and facsimile number or transmission address for the user-selected pharmacy. The most common method of transmission to pharmacies is via facsimile, however, it would be well within the skill of the art of the current invention to configure the calamity averse prescription engine 20 to request and receive transmission information for e-mail or other similar electronic methods. An example illustration of the patient 10 and pharmacy 6 information presentation can be seen in FIG. 11.

After receiving the patient name, date of birth and phone and facsimile numbers, the calamity averse prescription engine 20 at box 900 then generates a pharmaceutical prescription comprising the patient's name, date of birth and phone number, the pharmaceutical therapeutic accepted by the patient 10 or user 9, the dose, dosage and refill information for the pharmaceutical therapeutic from the acute symptom therapy table, phone number and facsimile number of the user-selected pharmacy, and a selected prescribing professional's name, address and NPI number. An example illustration of the pharmaceutical prescription is shown in FIG. 13. Once generated, the pharmaceutical prescription is then transmitted to the user-selected pharmacy via facsimile or other electronic method by the calamity averse prescription engine 20 at box 900.

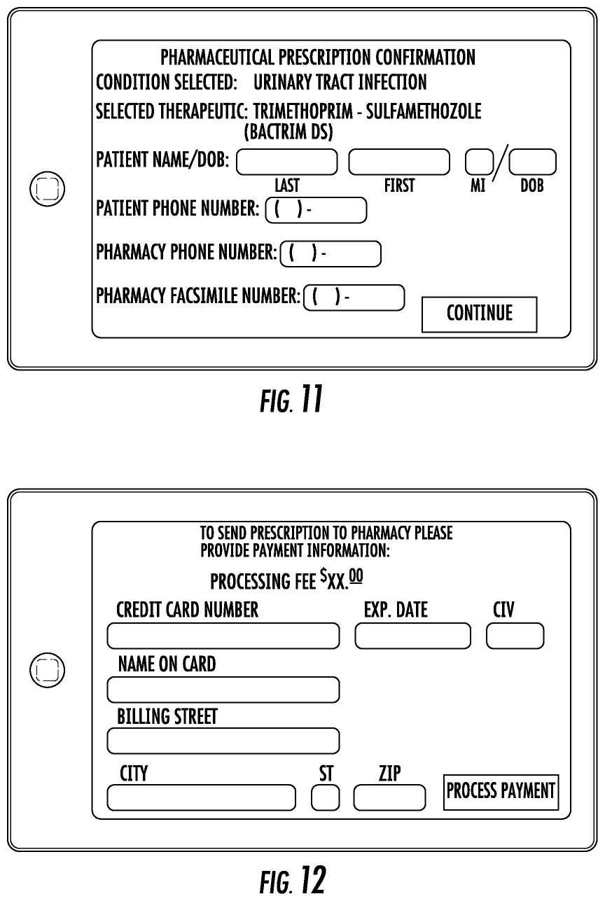

In the preferred embodiment, the patient 10 or user 9 may be presented with the option of free use of the calamity averse prescription apparatus 1 or requirement of a necessary processing fee in order to continue use of the apparatus 1. In this regard, the calamity averse prescription engine 20 at box 800, or at any point prior to box 900, presents a processing fee requirement to the patient 10 or user 9 and receives processing fee payment information from the patient 10 or user 9 prior to generating and transmitting the pharmaceutical prescription in box 900. An example of the processing fee requirement and processing fee payment information presentation is illustrated in FIG. 12

A pharmaceutical prescription must be authorized by a prescribing professional. In order to accommodate multiple prescribing professionals, each prescribing professional possibly having different preferences regarding conditions and prescriptions, the calamity averse prescription engine 20 refers to the prescribing professional credential table 50 to match the desired pharmaceutical therapeutic with an associated prescribing professional. In the case where there are multiple matches, the calamity averse prescription engine 20 is capable of multiple modes of information retrieval from the prescribing professional credential table 50. For instance, when multiple prescribing professionals are associated with a single pharmaceutical therapeutic, the calamity averse prescription engine 20 at box 900 may select a prescribing professional based on a rotating, fixed, random or other methodology.

Finally, in box 1000, the calamity averse prescription engine 20 creates the electronic medical record by storing as a single record in the electronic medical record database 60, the patient name, date of birth (DOB), address, phone number, the terms of use and disclaimer acknowledgements, the selected condition, the advisory acknowledgements, the selected pharmaceutical therapeutic and associated dose, dosage and refill information, the prescribing professional's name, address and NPI number, and the pharmacy phone number and facsimile number, then proceeds to END.

What is claimed is:

1. A method of issuing a pharmaceutical prescription for a patient to a pharmacy for treating a condition resulting from a common, acute, simple pathological process comprising;
presenting to a user computing device a plurality of conditions, wherein each of the plurality of conditions is associated with both a common, acute, simple pathological process and with at least one possible emergent, urgent or complicated condition;
receiving from the user computing device a selected condition from the plurality of conditions;
presenting to the user computing device at least one advisory associated with each of the at least one possible emergent, urgent or complicated condition for the selected condition;
receiving from the user computing device an advisory acknowledgement for each of the at least one advisory presented;
presenting to the user computing device at least one pharmaceutical therapeutic for treating the common, acute, simple pathological process, wherein the each of the at least one pharmaceutical therapeutic is associated with the selected condition and has an associated dose, dosage and refill number;
receiving from the user computing device a selected pharmaceutical therapeutic from the at least one pharmaceutical therapeutic presented;
receiving from the user computing device the patient name, date of birth, phone number, and a pharmacy transmission address;
creating the pharmaceutical prescription wherein a prescribing professional has pre-approved issuing a pharmaceutical prescription for the selected pharmaceutical therapeutic for treating the associated common, acute, simple pathological process;
transmitting the pharmaceutical prescription to the pharmacy using the pharmacy transmission address; and
creating and storing an electronic medical record.

2. The method of claim 1 wherein presenting to the user computing device at least one advisory associated with the at least one possible emergent, urgent or complicated condition for the selected condition comprises:
presenting to the user computing device at least one disqualifying co-condition, at least one risk associated with the at least one disqualifying co-condition, and at least one possible cause of the at least one disqualifying co-condition.

3. The method of claim 1 further comprising presenting to the user computing device a terms-of-use and a disclaimer governing use of the method; and receiving from the user computing device a terms-of-use acknowledgement and a disclaimers acknowledgement.

4. The method of claim 1 further comprising presenting to the user computing device a processing fee for use of the method, and receiving from the user computing device processing fee payment information for use of the method.

5. The method of claim 1 wherein the prescribing professional has an associated name, address and NPI number.

6. The method of claim 5 wherein creating the pharmaceutical prescription comprises compiling
the received patient name, date of birth, phone number, selected pharmaceutical therapeutic and pharmacy transmission address;
the dose, dosage and refill number associated with the pharmaceutical therapeutic; and
the name, address, phone number and NPI number associated with the prescribing professional associated with the pharmaceutical therapeutic into a single document.

7. The method of claim 6 wherein creating and storing an electronic medical record comprises creating in a single record:
the patient name, the date of birth, the patient phone number, the selected condition, the advisory acknowledgement for each of the at least one advisory presented, the selected pharmaceutical therapeutic and associated dose, dosage and refill information, the pharmacy transmission address, the prescribing professional name, address, phone number and NPI number; and the date and time of the transmission,
and storing the single record in the electronic medical record database.

8. An apparatus for issuing a pharmaceutical prescription for a patient to a user-selected pharmacy for treating a condition resulting from a common, acute, simple pathological process comprising:
a server connected to a network, the server being in communication with or comprising:
at least one processor and memory;
at least one non-volatile memory; and
a calamity adverse prescriptive engine,
wherein the at least one non-volatile memory comprises:
a calamitous symptom table comprising a plurality of conditions, each condition associated with both a common, acute, simple pathological process, and with at least one possible emergent, urgent or complicated condition, each of the at least one possible emergent, urgent or complicated condition comprising at least one advisory, each of the at least one advisory comprising a disqualifying possibly calamitous co-condition, a risk of disqualifying co-condition, and a cause of disqualifying co-condition;
an acute symptom therapy table comprising at least one pharmaceutical therapeutic associated with each condition;
a prescribing professional credential table comprising at least one prescribing professionals name, address, phone number, NPI number and at least one pharmaceutical therapeutic associated with the at least one prescribing professional; and
an electronic medical record database,
and wherein the calamity adverse prescriptive engine comprises:
an advisory confirmation module configured to present to the user computing device at least one advisory associated with the at least one possible emergent, urgent or complicated condition for a selected condition, and receive from the user computing device an advisory acknowledgement for each of the at least one advisory presented; and
a prescription confirmation module configured to present to the user computing device at least one pharmaceutical therapeutic for treating the common, acute, simple pathological process, and receive from the user computing device a selected pharmaceutical therapeutic from the at least one presented pharmaceutical therapeutic,
wherein the calamity adverse prescriptive engine is configured to present to the user computing device the plurality of conditions, receive from the user computing device the selected condition from the plurality of conditions, receive from the advisory confirmation module the advisory acknowledgement for the each of the at least one advisory presented, receive from the prescription confirmation module the selected pharmaceutical therapeutic, receive from the user computing device a patient name, date of birth, phone number, and pharmacy transmission address, generate and transmit a pharmaceutical prescription to the user-selected pharmacy using the pharmacy transmission address, and save an electronic medical record to the electronic medical record database.

9. The apparatus of claim 8, wherein the acute symptom therapy table further comprises a dose and a dosage associated with each pharmaceutical therapeutic.

10. The apparatus of claim 8 wherein the calamity adverse prescriptive engine is configured to generate pharmaceutical prescription to include the patent name, the selected pharmaceutical therapeutic, the prescribing professional's name, address, phone number and NPI number.

11. The apparatus of claim 8 wherein the calamity adverse prescriptive engine is configured to save the electronic medical record comprising the patient name, the selected condition, the advisory acknowledgement for each of the at least one advisory presented, the selected pharmaceutical therapeutic, and the pharmacy transmission address.

12. The apparatus of claim 8 wherein the calamity adverse prescriptive engine is further configured to present a terms-of-use and a disclaimer governing use of the method to the user computing device; and to receive a terms-of-use acknowledgement and a disclaimers acknowledgement from the user computing device.

13. The apparatus of claim 8 wherein the calamity adverse prescriptive engine is further configured to present a processing fee for use of the method to the user computing device, and receive processing fee payment information for use of the method from the user computing device.

14. The apparatus of claim 8 further comprising at least one input/output (I/O) interface.

15. A non-transitory computer-readable media having stored thereon instructions executable by a computing system including at least one server, wherein execution of the instructions implements a method of issuing a pharmaceutical prescription for a patient to a user-selected pharmacy for treating a condition resulting from a common, acute, simple pathological process comprising:
presenting to a user computing device a plurality of conditions, wherein each of the plurality of conditions is associated with both a common, acute, simple pathological process and with at least one possible emergent, urgent or complicated condition;
receiving from the user computing device a selected condition from the plurality of conditions;
presenting to the user computing device at least one advisory associated with each of the at least one possible emergent, urgent or complicated condition for the selected condition;
receiving from the user computing device an advisory acknowledgement for each of the at least one advisory presented;
presenting to the user computing device at least one pharmaceutical therapeutic for treating the common, acute, simple pathological process, wherein the each of the at least one pharmaceutical therapeutic is associated with the selected condition and has an associated dose, dosage and refill number;
receiving from the user computing device a selected pharmaceutical therapeutic from the at least one pharmaceutical therapeutic presented;
receiving from the user computing device the patient name, date of birth, phone number, and a pharmacy transmission address;
creating the pharmaceutical prescription wherein a prescribing professional has pre-approved issuing a pharmaceutical prescription for the selected pharmaceutical therapeutic for treating the associated common, acute, simple pathological process;
transmitting the pharmaceutical prescription to the pharmacy using the pharmacy transmission address; and
creating and storing an electronic medical record.
